(19) United States
(12) Patent Application Publication
(10) Pub. No.: US 2016/0048959 A1
(43) Pub. Date: Feb. 18, 2016

(54) Classifying Image Data for Vasospasm Diagnosis

(71) Applicants: Markus Kowarschik, Nurnberg (DE); Stefan Lautenschläger, Nurnberg (DE)

(72) Inventors: Markus Kowarschik, Nurnberg (DE); Stefan Lautenschläger, Nurnberg (DE)

(21) Appl. No.: 14/460,132

(22) Filed: Aug. 14, 2014

(51) Int. Cl. G06T 7/00; A61B 6/00; G06T 15/08; G06T 11/00; A61B 6/02; G06T 1/00

(52) U.S. Cl. CPC G06T 7/0014; A61B 6/027; G06T 1/0007; G06T 15/08; G06T 11/005; A61B 6/5229; A61B 6/481; A61B 6/504; A61B 6/4441; G06T 2211/404; G06T 2219/012; G06T 2207/30016; G06T 2207/30104

(57) ABSTRACT

In order to classify a cerebral vascular segment as normal or pathological, a time-series of three dimensional (3D) images representing the cerebral vascular segment is generated. A length of the cerebral vascular segment is determined, and a blood flow speed through the cerebral vascular segment is determined based on the length and the generated time-series of 3D images. The cerebral vascular segment is categorized based on the determined blood flow, and a representation of the cerebral vascular segment is displayed based on the categorization.

### FIELD

The present embodiments relate to classifying image data for vasospasm diagnosis.

### BACKGROUND

Vasospasm (e.g., angiospasm) is a sudden cramp-like constriction of a blood vessel (e.g., an arterial vessel) caused by an irritation. Vasospasm may lead to ischemia (e.g., inadequate perfusion) of tissue downstream of the arterial vessel.

Cerebral vasospasms are a frequent and serious complication of subarachnoid bleeding. Cerebral vasospasms also occur in other neurological diseases, in certain instances of poisoning (e.g., ergotism), as a result of medical procedures (e.g., angiographic therapies/interventions), as a side effect of medications, and in conjunction with the taking of drugs (e.g., cocaine and methamphetamines). For a proximal vasospasm, transcranial Doppler sonography methods may be used to detect the existence of the vasospasm.

### SUMMARY

In order to classify a cerebral vascular segment as normal or pathological, a time-series of three dimensional (3D) images representing the cerebral vascular segment is generated. A length of the cerebral vascular segment is determined, and a blood flow speed through the cerebral vascular segment is determined based on the length and the generated time-series of 3D images. The cerebral vascular segment is categorized based on the determined blood flow, and a representation of the cerebral vascular segment is displayed based on the categorization.

In a first aspect, a method for classifying image data representing a volume is provided. The method includes generating, by an imaging device, a plurality of 2D datasets. The plurality of 2D datasets represents the volume with the contrast medium injected into the volume. A processor generates a 3D dataset representing the volume based on the plurality of 2D datasets. The processor generates a time-series of 3D images of the volume based on the 3D dataset representing the volume, and the plurality of 2D datasets. The method includes determining a length of a portion of the 3D dataset, and determining a speed of blood flow within the volume based on the generated time-series of 3D images of the volume and the determined length of the portion of the 3D dataset.

In a second aspect, a non-transitory computer-readable storage medium that stores instructions executable by one or more processors for vasospasm diagnosis is provided. The instructions include generating 2D digital subtraction angiography (DSA) image data representing a volume of a patient from a number of directions around the volume based on 2D fill image data and 2D mask image data. The volume includes one or more arteries of the patient. The instructions also include generating 3D constraining image data based on the 2D DSA image data, and generating a time-series of 3D image datasets. The generating of the time-series of 3D image datasets includes combining the 3D constraining image data with the 2D DSA image data. The instructions include determining a length of an artery of the one or more arteries represented within the 3D constraining image data, respectively. The instructions also include determining a blood flow speed through the artery represented within the 3D constraining image data based on the time-series of 3D image datasets and the determined length of the artery. The instructions include identifying vasospasm within the volume of the patient based on the determined blood flow speed through the artery.

In a third aspect, a system for classifying data representing a volume of a patient is provided. The system includes an imaging device configured to generate first 2D datasets. The first 2D datasets represent the volume without a contrast medium injected into the volume from a number of directions relative to the volume. The imaging device is further configured to generate second 2D datasets. The second 2D datasets represent the volume with the contrast medium injected into the volume from the number of directions relative to the volume. The system also includes a processor configured to generate 2D DSA datasets. The generation of the 2D DSA datasets includes subtraction of the first 2D datasets from the second 2D datasets, respectively. The processor is also configured to reconstruct a 3D dataset representing the volume based on the 2D DSA datasets. The processor is configured to generate a time-series of 3D images of the volume. The generation of the time-series of 3D images of the volume includes a back-projection of the 2D DSA datasets into the 3D dataset. The processor is configured to determine a length of a portion of the 3D dataset. The processor is further configured to determine a blood flow speed through the portion of the volume based on the generated time-series of 3D images of the volume and the determined length of the portion of the 3D dataset. The system includes a display configured to display a representation of the reconstructed 3D dataset representing the volume. The display is also configured to visually categorize the blood flow speed through the portion of the volume on the displayed representation of the reconstructed 3D dataset.

### BRIEF DESCRIPTION OF THE DRAWINGS

FIG. 1 shows one embodiment of an imaging system;

FIG. 2 shows an imaging system including one embodiment of an imaging device; and

FIG. 3 shows a flowchart of one embodiment of a method for classifying image data for vasospasm diagnosis.

### DETAILED DESCRIPTION

Classification of whether flow speeds within three dimensional (3D) cerebral vascular segments are normal or pathological is provided. A 3D image of a cerebral vascular tree is reconstructed (e.g., a 3D view of the vessels without any dynamic information regarding blood flow) based on 2D projections generated by an imaging system. Combined 3D+T datasets (e.g., three spatial dimensions plus the dimension of time) are generated based on the 3D image of the cerebral vascular tree and 2D projections used to generate the 3D image. Blood flow in the cerebral vascular tree may be determined from and displayed with the 3D+T datasets.

Data representing main arteries is segmented from the 3D cerebral vascular tree to define arterial segments. Lengths of the arterial segments are determined based on the segmented data. Based on the determined lengths and the sufficiently high chronological resolution of the 3D+T datasets, the blood flow speed in each of the arterial segments may be determined by estimating transit times of the contrast agent bolus. Bolus transit times may be estimated by measuring time/contrast curves at various positions in the vascular tree and determining the associated transit times by cross-correlation.

The 3D image may be color coded in accordance with the determined blood flow speeds. As an example, portions of the 3D image corresponding to blood flow speeds of less than 140 cm/s, for example, may be colored green, which indicates no vasospasm. Portions of the 3D image corresponding to blood flow speeds between 140 cm/s and 200 cm/s, inclusive, for example, may be colored yellow, which indicates suspected vasospasm. Portions of the 3D image corresponding to blood speeds greater than 200 cm/s, for example, may be colored red, which indicates severe vasospasm.

Since suspicious areas and vasospasms are automatically visualized, reliable vasospasm detection is provided. This detection contributes to the medical success of therapy for the patient.

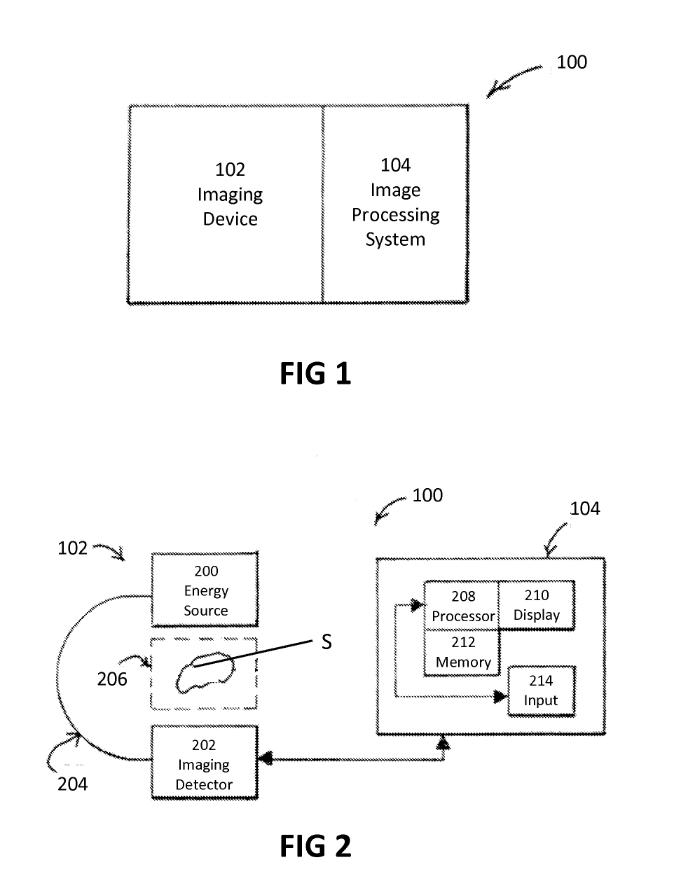

FIG. 1 shows one embodiment of an imaging system 100. The imaging system 100 is representative of an imaging modality. The imaging system 100 includes one or more imaging devices 102 and an image processing system 104. A two-dimensional (2D) or a three-dimensional (3D) (e.g., volumetric) image dataset may be acquired using the imaging system 100. The 2D image data set or the 3D image data set may be obtained contemporaneously with the planning and execution of a medical treatment procedure or at an earlier time. Additional, different, or fewer components may be provided.

The imaging device 102 includes a C-arm X-ray device (e.g., a C-arm angiography X-ray device). In one embodiment, the imaging device 102 is a biplane Artis dBA system or an Artis Zeego flat detector angiographic system (e.g., Dyna4D™). Alternatively or additionally, the imaging device 102 may include a gantry-based X-ray system, a magnetic resonance imaging (MRI) system, an ultrasound system, a positron emission tomography (PET) system, a single photon emission computed tomography (SPECT) system, a fluoroscopy, another X-ray system, any other now known or later developed imaging systems, or any combination thereof.

The image processing system 104 is a workstation, a processor of the imaging device 102, or another image processing device. The imaging system 100 may be used to generate a time-series of 3D images of a volume of a patient including one or more arteries, and to determine one or more blood flow speeds through the one or more arteries, respectively, based on the time-series of 3D images of the volume of the patient. For example, the image processing system 104 is a workstation for generating the time-series of 3D images of the volume and determining the one or more blood flow speeds. The time-series of 3D images of the volume may be generated from data generated by the one or more imaging devices 102 (e.g., a C-arm angiography device or a CT device). The workstation 104 receives data representing the volume generated by the one or more imaging devices 102.

FIG. 2 shows one embodiment of the imaging system 100 including the imaging device 102. The imaging device 102 is shown in FIG. 2 as a C-arm X-ray device. The imaging device 102 may include an energy source 200 and an imaging detector 202 connected together by a C-arm 204. Additional, different, or fewer components may be provided. In other embodiments, the imaging device 102 may be, for example, a gantry-based CT device.

The energy source 200 and the imaging detector 202 may be disposed opposite each other. For example, the energy source 200 and the imaging detector 202 are disposed on diametrically opposite ends of the C-arm 204. Arms of the C-arm 204 may be configured to be adjustable lengthwise. In certain embodiments, the C-arm 204 may be movably attached (e.g., pivotably attached) to a displaceable unit. The C-arm 204 may be moved on a buckling arm robot or other support structure. The robot arm allows the energy source 200 and the imaging detector 202 to move on a defined path around the patient. During acquisition of non-contrast and contrast scans, for example, the C-arm 204 is swept around the patient. During the contrast scans, contrast agent may be injected intravenously. In another example, the energy source 200 and the imaging detector 202 are connected inside a gantry.

The energy source 200 may be a radiation source such as, for example, an X-ray source. The energy source 200 may emit radiation to the imaging detector 202. The imaging detector 202 may be a radiation detector such as, for example, a digital-based X-ray detector or a film-based X-ray detector. The imaging detector 202 may detect the radiation emitted from the energy source 200. Data is generated based on the amount or strength of radiation detected. For example, the imaging detector 202 detects the strength of the radiation (e.g., intensity) received at the imaging detector 202 and generates data based on the strength of the radiation. The data may be considered imaging data as the data is used to then generate an image. Image data may also include data for a displayed image.

During each rotation, the C-arm X-ray device 102 may acquire between 50-500 projections, between 100-200 projections, or between 100-150 projections. In other embodiments, during each rotation, the C-arm X-ray device 102 may acquire between 50-100 projections per second, or between 50-75 projections per second. Any speed, number of projections, dose levels, or timing may be used.

A region 206 to be examined (e.g., a volume; the brain of a patient) is located between the energy source 200 and the imaging detector 202. The region 206 to be examined may include one or more structures S (e.g., one or more volumes of interest or one or more arteries), through which the blood flow speed is to be calculated. The region 206 may or may not include a surrounding area. For example, the region 206 to be examined may include the brain and/or other organs or body parts in the surrounding area of the brain.

The data generated by the one or more imaging devices 102 and/or the image processing system 104 may represent (1) a projection of 3D space to 2D or (2) a reconstruction (e.g., computed tomography) of a 3D region from a plurality 2D projections (e.g., (1) 2D data or (2) 3D data, respectively). For example, the C-arm X-ray device 102 may be used to obtain 2D data or CT-like 3D data. A computer tomography (CT) device may obtain 2D data or 3D data. The data may be obtained from different directions. For example, the imaging device 102 may obtain data representing sagittal, coronal, or axial planes or distribution.

The imaging device 102 may be communicatively coupled to the image processing system 104. The imaging device 102 may be connected to the image processing system 104, for example, by a communication line, a cable, a wireless device, a communication circuit, and/or another communication device. For example, the imaging device 102 may communicate the data to the image processing system 104. In another example, the image processing system 104 may communicate an instruction such as, for example, a position or angulation instruction to the imaging device 102. All or a portion of the image processing system 104 may be disposed in the imaging device 102, in the same room or different rooms as the imaging device 102, or in the same facility or in different facilities. The image processing system 104 may represent a plurality of image processing systems associated with more than one imaging device 102. In alternative embodiments, the imaging device 102 communicates with an archival system or memory. The image processing system 104 retrieves or loads the 2D or 3D data from the memory for processing.

In the embodiment shown in FIG. 2, the image processing system 104 includes a processor 208, a display 210 (e.g., a monitor), and a memory 212. Additional, different, or fewer components may be provided. For example, the image processing system 104 may include an input device 214, a printer, and/or a network communications interface.

The processor 208 is a general processor, a digital signal processor, an application specific integrated circuit, a field programmable gate array, an analog circuit, a digital circuit, another now known or later developed processor, or combinations thereof. The processor 208 may be a single device or a combination of devices such as, for example, associated with a network or distributed processing. Any of various processing strategies such as, for example, multi-processing, multi-tasking, and/or parallel processing may be used. The processor 208 is responsive to instructions stored as part of software, hardware, integrated circuits, firmware, microcode or the like.

The processor 208 may generate an image from the data. The processor 208 processes the data from the imaging device 102 and generates an image based on the data. For example, the processor 208 may generate one or more angiographic images, fluoroscopic images, top-view images, in-plane images, orthogonal images, side-view images, 2D images, 3D representations or images (e.g., renderings or volumes from 3D data to a 2D display), progression images, multi-planar reconstruction images, projection images, or other images from the data. In another example, a plurality of images may be generated from data detected from a plurality of different positions or angles of the imaging device 102 and/or from a plurality of imaging devices 102.

The processor 208 may generate a 2D image from the data. The 2D image may be a planar slice of the region 206 to be examined. For example, the C-arm X-ray device 102 may be used to detect data representing voxels of a 3D volume, from which a sagittal image, a coronal image, and an axial image are extracted along a plane. The sagittal image is a side-view image of the region 206 to be examined. The coronal image is a front-view image of the region 206 to be examined. The axial image is a top-view image of the region 206 to be examined.

The processor may generate a 3D representation or image from the data. The 3D representation illustrates the region 206 to be examined. The 3D representation may be generated from a reconstructed volume (e.g., by combining 2D datasets, such as with computed tomography) obtained by the imaging device 102. For example, a 3D representation may be generated by analyzing and combining data representing different planes through the patient, such as a stack of sagittal planes, coronal planes, and/or axial planes, or a plurality of planes through the patient at different angles relative to the patient. Additional, different, or fewer images may be used to generate the 3D representation. Generating the 3D representation is not limited to combining 2D images. For example, any now known or later developed method may be used to generate the 3D representation.

The processor 208 may display the generated images on the monitor 210. For example, the processor 208 may generate the 3D representation and communicate the 3D representation to the monitor 210. The processor 208 and the monitor 210 may be connected by a cable, a circuit, another communication coupling or a combination thereof. The monitor 210 is a monitor, a CRT, an LCD, a plasma screen, a flat panel, a projector or another now known or later developed display device. The monitor 210 is operable to generate images for a two-dimensional view or a rendered three-dimensional representation. For example, a two-dimensional image representing a three-dimensional volume through projection or surface rendering is displayed.

The processor 208 may communicate with the memory 212. The processor 208 and the memory 212 may be connected by a cable, a circuit, a wireless connection, another communication coupling, or any combination thereof. Images, data, and other information may be communicated from the processor 208 to the memory 212 for storage, and/or the images, the data, and the other information may be communicated from the memory 212 to the processor 208 for processing. For example, the processor 208 may communicate the generated images, image data, or other information to the memory 212 for storage.

In one embodiment, the processor 208 is programmed to generate 2D digital subtraction angiography (DSA) datasets and reconstruct a 3D dataset representing a volume (e.g., a portion of a brain) based on the 2D DSA datasets. The processor 208 may be further programmed to generate a time-series of 3D images of the volume, determine a length of a region within the volume, and determine a blood flow speed through the region based on the time-series of 3D images and the determined length.

The memory 212 is a non-transitory computer readable storage media. The computer readable storage media may include various types of volatile and non-volatile storage media, including but not limited to random access memory, read-only memory, programmable read-only memory, electrically programmable read-only memory, electrically erasable read-only memory, flash memory, magnetic tape or disk, optical media and the like. The memory 212 may be a single device or a combination of devices. The memory 212 may be adjacent to, part of, networked with and/or remote from the processor 208.

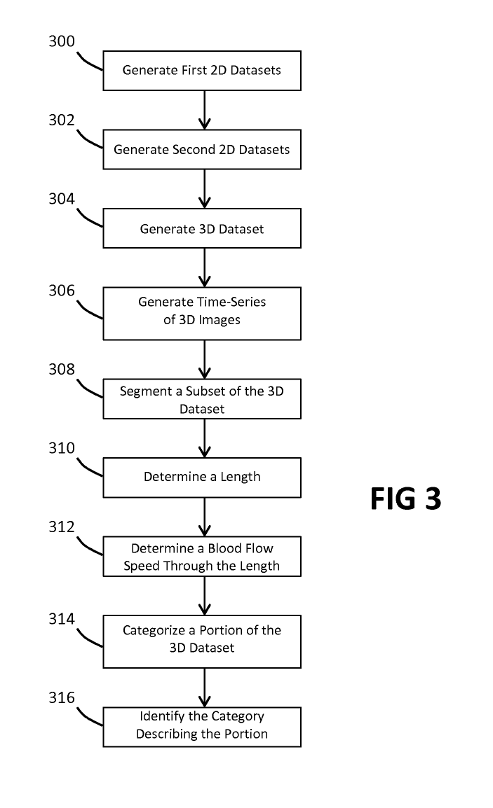

FIG. 3 shows a flowchart of one embodiment of a method for classifying image data representative of a volume. The image data may, for example, be computed tomography (CT) image data or image data generated during rotation of a C-arm during X-ray imaging. The method may be performed using the imaging system 100 shown in FIGS. 1 and 2 (e.g., at least some of the acts of the method may be performed by the processor 208) or another imaging system. For example, the acts of the method are implemented by one or more processors using instructions from one or more memories. The method is implemented in the order shown, but other orders may be used. Additional, different, or fewer acts may be provided. Similar methods may be used for classifying image data.

In act 300, an imaging device generates a plurality of first 2D datasets. The plurality of first 2D datasets represents the volume without a contrast medium injected into the volume. The volume may represent at least a portion of a patient and may include, for example, the brain of the patient. The volume may also include tissue, bone, and air surrounding the brain of the patient. In other embodiments, the volume includes one or more other or different body parts or organs of the patient.

In one embodiment, the imaging device is a C-arm X-ray device. Other imaging devices (e.g., a CT device) may be used. The C-arm X-ray device generates the plurality of first 2D datasets by generating a plurality of first projections into the volume over an angular range. These first 2D datasets are acquired without contrast agent injected into the patient. The C-arm X-ray device may generate any number of projections over the angular range. The projections may be generated over one or more rotations in the same or alternating directions. The angular range may be an angular range of a C-arm of the C-arm X-ray device. Alternatively, the angular range may be, for example, an angular range of a gantry of the CT device. The angular range may, for example, be 200° in a forward rotation of the C-arm X-ray device. In other embodiments, the C-arm X-ray device generates projections over a different angular range and/or in a different direction. A speed of the angular rotation of the C-arm X-ray device, for example, may vary based on the application. For example, the C-arm X-ray device may be rotated through the angular range in 6 s when arteries are to be imaged, and may be rotated through the angular range in 12 s when arteries and veins are to be imaged.

In other embodiments, the plurality of first 2D datasets are generated at fixed angles (e.g., no rotational sweeps, separate acquisitions for 2D and 3D data, and separate contrast agent injections), with a monoplane acquisition, and/or with a biplane acquisition.

The plurality of first 2D datasets may be stored in a memory in communication with a processor. Alternatively or additionally, the processor generates and/or further processes the plurality of first 2D datasets based on data received from the C-arm X-ray device. In another embodiment, the processor identifies previously generated and stored first 2D datasets.

In act 302, the imaging device generates a plurality of second 2D datasets. Each second 2D dataset of the plurality of second 2D datasets represents a projection of the volume with the contrast medium injected into the volume. The contrast agent may be administered to or injected into the patient either venously or arterially. In one embodiment, the processor generates and/or further processes the plurality of second 2D datasets based on data received from the C-arm X-ray device, for example. The plurality of second 2D datasets may be generated a short (e.g., 10 s) or a long (e.g., one day, one week) time period after the plurality of first 2D datasets are generated.

The C-arm X-ray device generates the plurality of second 2D datasets by generating a plurality of second projections with the contrast agent injected into the volume over the angular range used for the first 2D dataset or a different angular range. The C-arm X-ray device may generate the plurality of second 2D datasets in a same direction of rotation of the C-arm or an opposite direction of rotation of the C-arm compared to during the generation of the plurality of first 2D datasets. The projections may be generated over one or more rotations in the same or alternating directions. The plurality of second 2D datasets may be generated in any number of acquisition times including, for example, 5 s, 8 s, and 10 s. For example, the C-arm X-ray device may be rotated through the angular range in 6 s when arteries are to be imaged, and may be rotated through the angular range in 12 s when arteries and veins are to be imaged. As another example, a 5 s acquisition may be provided for evaluation of a patient with an aneurysm at the circle of Willis or a fast-flow carotid cavernous fistula, and a 8 s or 10 s acquisition may be provided for a patient with occlusive disease in which filling occurs by collaterals. The acquisition time for the plurality of first 2D datasets may be the same as or different than the acquisition time for the plurality of second 2D datasets. A rotational speed of the C-arm X-ray device, for example, may set the acquisition time. The acquisition time may be set to capture a full cycle of contrast inflow and washout (e.g., long enough to follow a bolus through vasculature).

The plurality of second 2D datasets may be stored in the memory or a different memory. Alternatively or additionally, the processor generates and/or further processes the plurality of second 2D datasets based on data received from the C-arm X-ray device. In another embodiment, the processor identifies previously generated and stored second 2D datasets.

In act 304, the processor generates a 3D dataset that represents the volume based on the plurality of first 2D datasets and the plurality of second 2D datasets. The processor may use all or some of the first 2D datasets and/or all or some of the second 2D datasets to generate the 3D dataset. The 3D dataset may be a 3D digital subtraction angiography (DSA) volume and may act as a constraining image (e.g., a max-fill volume). In one embodiment, the processor generates the 3D dataset that represents the volume based on data generated during a single rotational run. In such an embodiment, DSA is not used, as image processing techniques such as window leveling and bone segmentation/subtraction are applied to the data generated during the single rotational run to generate the 3D dataset.

In one embodiment, the processor registers the plurality of first 2D datasets with the plurality of second 2D datasets. The plurality of first 2D datasets may be registered with the plurality of second 2D datasets in any number of ways including, for example, using 2D-2D rigid registration based on comparison of the datasets. Other registration methods may be used. Other data sets may be used as the reference (i.e., register to a different data set). The registration spatially aligns the data sets to counter any motion that occurs between acquisitions of the data sets. The spatial transform for the registration may be rigid or non-rigid.

The processor may apply a filter to preserve edges around high contrast vessels within the plurality of second 2D datasets. In one embodiment, a non-smoothing Shepp-Logan filter kernel is used to preserve the edges. Other filters may be used.

The plurality of first 2D datasets (e.g., without contrast) are subtracted from the plurality of second 2D datasets (e.g., with contrast), respectively, to generate a plurality of 2D DSA datasets. The processor may store the plurality of 2D DSA datasets in the memory. In one embodiment, the X-ray detector of the C-arm X-ray device, for example, may be a counting detector (e.g., an energy discriminating detector) that may generate contrast-only images based on a single acquisition (i.e., no subtraction of corresponding pairs of images). In such an embodiment, DSA is not used, and only a single acquisition (e.g., of the plurality of second datasets) is used to generate the 3D dataset.

In one embodiment, the processor reconstructs the 3D DSA dataset based on the plurality of 2D DSA datasets using any number of reconstruction algorithms including, for example, the Feldkamp algorithm. The result of the reconstruction is a volumetric dataset representing X-ray attenuation values associated with a plurality of voxels representing the volume that has been imaged. The 3D DSA dataset represents a volume describing contrast agent enhancement since mask information (e.g., the first 3D dataset) is subtracted. The tissue or other non-contrast information is removed, leaving contrast information and any tissue with different attenuation and/or due to misregistration.

In one embodiment, the processor generates a first 3D dataset based on the plurality of first 2D datasets, and generates a second 3D dataset based on the plurality of second 2D datasets. The processor may reconstruct the first 3D dataset and the second 3D dataset using any number of reconstruction algorithms including, for example, the Feldkamp algorithm. Other reconstruction algorithms may be used.

The processor registers the first 3D dataset and the second 3D dataset. The registration spatially aligns the data sets to counter any motion that occurs between acquisitions of the data sets. The first 3D dataset and the second 3D dataset may be registered in any number of ways including, for example, using 3D-3D rigid registration. Other registration methods may be used. For example, the spatial transform for the registration may be non-rigid. Either the first 3D dataset or the second 3D dataset may be used as the reference for registration. The first 3D dataset and the second 3D dataset may be stored in the memory after the processor has generated the first 3D dataset and the second 3D dataset, respectively.

The processor may generate the 3D DSA dataset based on the first 3D dataset and the second 3D dataset. The processor may generate the 3D DSA dataset by subtracting the first 3D dataset from the second 3D dataset.

The 3D DSA dataset generated in act 304 does not have any time dependence, as the data used to generate the 3D DSA dataset (e.g., the plurality of first 2D datasets and the plurality of second 2D datasets) is averaged over a time period the C-arm X-ray device takes to move through the angular range (e.g., 12 s). The 3D DSA dataset represents a single vascular volume over the angular range.

In act 306, the processor generates a time-series of 3D images of the volume (e.g., a series of time-resolved 3D volumes; 4D-DSA) based on the 3D dataset generated in act 304 (e.g., the 3D DSA dataset or the constraining volume) and the plurality of 2D DSA datasets generated in act 304. The processor generates the time-series of 3D images using multiplicative projection processing (e.g., a 4D DSA method; Dyna4D), for example. Other techniques or algorithms may be used to generate the time-series of 3D images. The multiplicative projection processing includes embedding (e.g., backprojecting) the time-resolved data from the plurality of 2D DSA datasets into the constraining volume. The time-series of 3D images thus represents the same time period as the plurality of first 2D datasets and the plurality of second 2D datasets. In one embodiment, the processor generates 30 time-resolved 3D-DSA volumes per second rather than 1 3D-DSA volume per gantry rotation.

Prior to generation of the time-series of 3D images, individual 2D DSA datasets may be spatially convolved to increase signal to noise ratio (SNR). Each of the spatially convolved 2D DSA datasets forms a low spatial frequency mask that enhances portions of the constraining volume that are present at each point in time during acquisition of the 2D datasets. After a normalization step, the spatially convolved 2D DSA datasets provide proper projection weighting. As a result of the spatial convolution, the SNR of the individual timeframes is limited by the constraining volume SNR ratio and not by the SNR of individual projections.

When the plurality of 2D DSA datasets are back-projected into the constraining volume, projection values from overlapping vessels may cause the deposition of erroneous signal (e.g., an opacity shadow from opacified vessel to nonopacified vessel) into vessels in the constraining volume. To reduce this effect, for each timeframe, the processor performs an angular (e.g., temporal) search, looking for a range of time before and after the frame that is being projected. After this search, a minimum signal for each voxel is assumed to be due to the ray with a minimum degree of overlap. This value is assigned to the timeframe being processed.

In act 308, the processor segments a subset of data from the 3D DSA dataset generated in act 304. The segmented subset of data corresponds to a subset of voxels of the 3D DSA dataset. The subset of data may represent, for example, main arteries of the patient (e.g., at least M1 through M3 of the cerebral artery). The subset of data may represent more, less, or different portions of the patient.

The processor segments the subset of data automatically and/or based on input from a user of the C-arm X-ray device or another user via an input device (e.g., a mouse). For example, the processor may generate a representation of the 3D DSA dataset and display the representation to the user via a display. The user may identify a region represented within the 3D DSA dataset to be segmented (e.g., the subset of data, which corresponds to one or more arteries within the brain of the patient) using the input device, and the processor may segment the representation of the one or more arteries based on the identified region received from the input device. As another example, the processor may automatically determine boundaries of the one or more arteries (e.g., based on changes in values of data within the 3D DSA dataset) and automatically segment data representing the one or more arteries from the 3D DSA dataset. With the segmentation of the one or more arteries, for example, a 3D course of the one or more arteries (e.g., arterial segments) is defined.

In act 310, the processor determines a length within the region represented by the segmented subset of data. For example, the processor determines a length of an artery of the one or more arteries represented by the segmented subset of data. The length may be between branches, across multiple branches, arbitrary, or as defined for a standard approach to artery segmentation or vasospasm processing. The processor may also determine lengths of additional arteries of the one or more arteries.

The user may identify a centerline of the artery using the input device (e.g., the mouse), for example. For example, the processor may generate an image representing the segmented subset of data and display the image on the display. The user may use the input device to define center points along the artery, and the processor may generate corresponding lines connecting the defined center points. As another example, the processor may automatically determine the centerline of the artery based on the automatically determined boundaries of the one or more arteries, skeletonization, or region shrinking. The centerline of the artery may be identified in different ways.

The processor may determine the length of the centerline based on the known dimensions the 3D DSA dataset represents. For example, the field of view of the C-arm X-ray device (e.g., based on the size of the detector of the C-arm X-ray imaging device) may define dimensions the 3D DSA dataset represents. The processor may determine the length of the centerline based on the dimensions the 3D DSA dataset represents and geometric principles. The length of the centerline may be determined in other ways.

In act 312, a blood flow speed through the length is determined. For example, the processor determines a time period contrast takes to move through the length (e.g., contrast flow time period) based on the time-series of 3D images of the volume generated in act 306. Each 3D image of the time-series of 3D images has a time that corresponds to the 3D image. As an example, the injected contrast may be at a start of the length in, for example, a fourth 3D image of the time-series of 3D images, and the start of the injected contrast may have flowed to an end of the length in, for example, a twentieth 3D image of the time-series of 3D images. The processer may determine the contrast flow time period based on a difference between the respective times represented by the fourth 3D image and the twentieth 3D image, for example. The processor may then calculate the speed of blood flow by dividing the length determined in act 310 by the contrast flow time period. Contrast flow time periods may be determined for a plurality of lengths (e.g., representing a plurality of arteries), and a plurality of blood flow speeds may thus be calculated.

The start of contrast is determined. In one embodiment, the processor creates curves of amount of contrast over time at various positions in the vascular tree and determines the associated transit times by cross-correlation. In another embodiment, at least some of the time-series of 3D images are displayed to the user, and the user identifies the 3D images of the time-series of 3D images that represent the image where the injected contrast is at the start of the length and the image where the injected contrast is at the end of the length (e.g., start and end 3D images), respectively, using the input device. Additionally or alternatively, the processor automatically identifies the start and end 3D images and presents the start and end 3D images to the user for verification. Interpolation may be used to more accurately determine the contrast flow time. For example, if one 3D image of the time-series of 3D image shows a front edge of the contrast flow before the start of the length, and the 3D image subsequent in time shows the front edge of the contrast flow after the start of the length, the time between the one 3D image and the subsequent 3D image (e.g., the time between scans) may be interpolated to determine a more accurate time at which the contrast reached the start of the length.

In act 314, the processor categorizes a portion of the 3D DSA dataset (e.g., the portion of the subset of data segmented from the 3D DSA dataset) based on the blood flow speed calculated in act 312. In one embodiment, a plurality of portions of the 3D DSA dataset are categorized based on a plurality of corresponding blood flow speeds calculated in act 312.

The processor categorizes the portion of the 3D DSA dataset based on one or more blood flow speed ranges and/or blood flow speed thresholds. For example, the processor may compare the blood flow speed calculated in act 312 to a first blood flow speed range, a second blood flow speed range, a first blood flow speed threshold, or any combination thereof, to determine a category describing the portion of the 3D DSA dataset.

The user may identify (e.g., set) the one or more blood flow speed ranges and/or blood flow speed thresholds using the input device, or the one or more blood flow speed ranges and/or blood flow speed thresholds may be predetermined and set within the imaging device (e.g., preprogrammed). The first blood flow speed range, the second blood flow speed range, and the first blood flow speed threshold, for example, may be stored in the memory. More or fewer blood flow speed ranges and/or blood flow speed thresholds may be identified and/or set. For example, only the first blood flow speed range and the second blood flow speed range are identified and/or set. As another example, a threshold speed separating normal flow from abnormal flow is set.

The first blood flow speed range may represent blood flow speeds at which no vasospasm is present. The second blood flow speed range may represent blood flow speeds at which vasospasm is suspected. The first blood flow speed threshold may represent a blood flow speed above which there is severe vasospasm. In one embodiment, any blood flow speed outside of the first blood flow speed range and the second blood flow speed range may be identified representing severe vasospasm. In one embodiment, the first blood flow speed range is 0 cm/s to 140 cm/s, exclusive, the second blood flow speed range is 140 cm/s, inclusive, to 200 cm/s, inclusive, and the first blood flow speed threshold is 200 cm/s.

In one embodiment, with increasing use of the method of FIG. 3 or other methods for categorizing blood flow speeds through vasculature, universal blood flow data may be generated. With the aid of data from a number of clinical sites and a number of patients, a statistically reliable database that identifies blood flow speeds considered physiologically normal and a blood flow speed at which the flow speed becomes pathological may be built up. With increasing knowledge, the first blood flow speed range, the second blood flow speed range, the first blood flow speed threshold, or a combination thereof may be optimized.

In act 316, the processor identifies, via the display, the category describing the portion of the 3D DSA dataset. For example, the processor displays, via the display, a representation of the 3D DSA dataset generated in act 304 and colors the portion of the 3D DSA dataset based on the category describing the portion of the 3D DSA dataset. For example, the processor colors the portion of the 3D DSA dataset green when the blood flow speed calculated in act 312 is within the first blood flow speed range, yellow when the blood flow speed calculated in act 312 is within the second blood flow speed range, and red when the blood flow speed calculated in act 312 is outside of the first blood flow speed range and the second blood flow speed range. Other colors may be used. The category describing the portion of the 3D DSA dataset may be identified, via the display, in any number of other ways including, for example, by labeling the portion of the 3D DSA dataset with the identified category. For example, the displayed portion of the 3D DSA dataset may be labeled with the text “VASOSPASM” when the blood flow speed calculated in act 312 is outside of the first blood flow speed range and the second blood flow speed range.

In one embodiment, a plurality of portions of the 3D DSA dataset are colored based on the categories describing the plurality of portions of the 3D DSA dataset, respectively. For example, a first artery represented within the 3D DSA dataset may be colored red, while a second artery and a third artery represented within the 3D DSA dataset may be colored green. More, fewer, and/or different distinctions and corresponding color codes may be provided. For example, a vasospasm may be further distinguished or classified by whether the vasospasm is slight, medium, or severe based on the calculated blood flow speed and additional blood flow speed ranges and/or blood flow speed thresholds.

In one embodiment, for portions of the 3D DSA dataset that represent spastic vascular portions, the processor may automatically search for vascular narrowings (e.g., stenoses) proximal to the portions of the 3D DSA dataset that represent spastic vascular portions. The processor may automatically analyze data representing arteries and/or vasculature proximal the portions of the 3D DSA dataset that represent spastic vascular portions using an embodiment of the method described above. For example, the user may identify, with the input device and/or the processor may identify the portions of the 3D DSA dataset that represent spastic vascular portions. The processor may identify a portion of the 3D DSA dataset that represents a spastic vascular portion based on a frequency of change of blood flow speed through the vascular portion. The user, with the input device, and/or the processor may identify data representing arteries and/or vasculature proximal to the spastic vascular portion to be analyzed using one embodiment of the method shown in FIG. 3.

The method shown in FIG. 3 may provide automatic analysis and color coding of a time-series of 3D images of a volume (e.g., a 3D+T dataset) without any further action on the part of the user (e.g., a physician). Because suspicious areas and vasospasms are automatically visualized, reliable vasospasm detection is provided, which contributes to the successfulness of therapy for a patient. Also, an implicit classification is automatically performed, and potential underlying constrictions/stenoses are automatically detected and measured.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for classifying image data representing a volume, the method comprising:
generating, by an imaging device, a plurality of two dimensional (2D) datasets, the plurality of 2D datasets representing the volume with a contrast medium injected into the volume;
generating, by a processor, a three dimensional (3D) dataset representing the volume based on the plurality of 2D datasets;
generating, by the processor, a time-series of 3D images of the volume based on the 3D dataset representing the volume, and the plurality of 2D datasets;
determining a length of a portion of the 3D dataset; and
determining a speed of blood flow within the volume based on the generated time-series of 3D images of the volume and the determined length of the portion of the 3D dataset.

2. The method of claim 1, further comprising:
generating, by the imaging device, a plurality of first 2D datasets, the plurality of first 2D datasets representing the volume without a contrast medium injected into the volume, wherein the plurality of 2D datasets are a plurality of second 2D datasets, wherein generating the 3D dataset representing the volume comprises generating the 3D dataset representing the volume based on the plurality of first 2D datasets and the plurality of second 2D datasets, and wherein generating the time-series of 3D images of the volume comprises generating the time-series of 3D images of the volume based on the 3D dataset representing the volume, the plurality of first 2D datasets, and the plurality of second 2D datasets;
generating a 3D image of the volume based on the generated 3D dataset;
displaying the 3D image of the volume; and
identifying a color associated with the determined speed of blood flow within the volume, wherein displaying the 3D image of the volume comprises displaying a portion of the 3D image corresponding to the portion of the 3D dataset in the identified color.

3. The method of claim 2, wherein the portion of the 3D dataset is a first portion of the 3D dataset, the speed of blood flow is a first speed of blood flow, and the color is a first color, and
wherein the method further comprises:
determining a length of a second portion of the 3D dataset;
determining a second speed of blood flow within the volume based on the generated time-series of 3D images of the volume and the determined length of the second portion of the 3D dataset; and
identifying a second color, the second color being associated with the determined second speed of blood flow within the volume, wherein displaying the 3D image of the volume comprises displaying a portion of the 3D image corresponding to the second portion of the 3D dataset in the identified second color.

4. The method of claim 3, further comprising segmenting the first portion of the 3D dataset and the second portion of the 3D dataset from the 3D dataset.

5. The method of claim 4, wherein the first portion of the 3D dataset and the second portion of the 3D dataset represent arteries of a patient, respectively.

6. The method of claim 3, further comprising identifying a first speed range corresponding to the first color and identifying a second speed range or a threshold corresponding to the second color.

7. The method of claim 6, wherein the identified first speed range is zero to 140 cm/s, and the first color is green, and
wherein the identified threshold is 200 cm/s, and the second color is red.

8. The method of claim 2, wherein generating the 3D dataset representing the volume comprises:
generating, by the processor, a plurality of third 2D datasets, the generating of the plurality of third 2D datasets comprising subtracting the plurality of first 2D datasets from the plurality of second 2D datasets, respectively; and
reconstructing the 3D dataset representing the volume based on the plurality of third 2D datasets.

9. The method of claim 6, wherein generating the time-series of 3D images of the volume comprises combining the 3D dataset representing the volume with each dataset of the plurality of third 2D datasets, the combining comprising back-projecting each dataset of the plurality of third 2D datasets into the 3D dataset representing the volume.

10. In a non-transitory computer-readable storage medium that stores instructions executable by one or more processors for vasospasm diagnosis, the instructions comprising:
generating two dimensional (2D) digital subtraction angiography (DSA) image data representing a volume of a patient from a number of directions around the volume, the volume including one or more arteries of the patient;
generating three dimensional (3D) constraining image data based on the 2D DSA image data;
generating a time-series of 3D image datasets, the generating of the time-series of 3D image datasets comprising combining the 3D constraining image data with the 2D DSA image data;
determining a length of an artery of the one or more arteries represented within the 3D constraining image data, respectively;
determining a blood flow speed through the artery represented within the 3D constraining image data based on the time-series of 3D image datasets and the determined length of the artery; and
identifying vasospasm within the volume of the patient based on the determined blood flow speed through the artery.

11. The non-transitory computer-readable storage medium of claim 10, wherein the instructions further comprise:
generating, with an imaging device, 2D mask image data representing the volume of the patient without a contrast medium injected into the patient from a number of directions around the volume; and
generating, with the imaging device, 2D fill image data representing the volume of the patient with the contrast medium injected into the patient from the number of directions around the volume, and
wherein generating the 2D DSA image data comprises generating the 2D DSA image data based on the 2D mask image data and the 2D fill image data.

12. The non-transitory computer-readable storage medium of claim 10, wherein the instructions further comprise identifying a first range of blood flow speeds, a second range of blood flow speeds, and a blood flow threshold indicating severe vasospasm, the first range of blood flow speeds being associated with no vasospasm, the second range of blood flow speeds being associated with suspected vasospasm, and blood flow speeds greater than the blood flow threshold being associated with severe vasospasm,
wherein identifying vasospasm within the volume of the patient comprises comparing the determined blood flow speed with the second range of blood flow speeds, the blood flow threshold, or a combination thereof.

13. The non-transitory computer-readable storage medium of claim 12, wherein the instructions further comprise:
displaying a representation of the 3D constraining image data, the displaying of the representation of the 3D constraining image data comprising displaying at least a portion of the artery within the representation of the 3D constraining image data in a first color when the determined blood flow speed is within the first range of blood flow speeds, in a second color when the determined blood flow speed is within the second range of blood flow speeds, and in a third color when the determined blood flow speed is greater than the blood flow threshold.

14. The non-transitory computer-readable storage medium of claim 10, wherein the instructions further comprise segmenting data representing the one or more arteries of the patient from the 3D constraining image data and the 2D DSA image data, and
wherein generating the time-series of 3D image datasets comprises combining the segmented data representing the one or more arteries of the patient from the 3D constraining image data with the segmented data representing the one or more arteries of the patient from the 2D DSA image data.

15. The non-transitory computer-readable storage medium of claim 10, wherein the combining comprises back-projecting the 2D DSA image data into the 3D constraining image dataset.

16. The non-transitory computer-readable storage medium of claim 10, wherein the determining of the blood flow speed through the artery comprises:
identifying a time period contrast takes to flow from a start of the determined length of the artery to an end of the determined length of the artery based on the time-series of 3D image datasets; and
dividing the determined length of the artery by the identified time period.

17. A system for classifying data representing a volume of a patient, the system comprising:
an imaging device configured to:
generate first two dimensional (2D) datasets, the first 2D datasets representing the volume without a contrast medium injected into the volume from a number of directions relative to the volume;
generate second 2D datasets, the second 2D datasets representing the volume with the contrast medium injected into the volume from the number of directions relative to the volume;
a processor configured to:
generate 2D digital subtraction angiography (DSA) datasets, the generation of the 2D DSA datasets comprising subtraction of the first 2D datasets from the second 2D datasets, respectively;
reconstruct a three dimensional (3D) dataset representing the volume based on the 2D DSA datasets;
generate a time-series of 3D images of the volume, the generation of the time-series of 3D images of the volume comprising a back-projection of the 2D DSA datasets into the 3D dataset;
determine a length of a portion of the 3D dataset; and
determine a blood flow speed through a portion of the volume based on the generated time-series of 3D images of the volume and the determined length of the portion of the 3D dataset; and
a display configured to:
display a representation of the reconstructed 3D dataset representing the volume; and
visually categorize the blood flow speed through the portion of the volume on the displayed representation of the reconstructed 3D dataset.

18. The system of claim 17, wherein the imaging device comprises a C-ram X-ray device.

19. The system of claim 17, wherein the display is configured to color a part of the representation of the reconstructed 3D dataset corresponding to the portion of the volume a first color corresponding to the determined blood flow speed, such that the blood flow speed through the portion of the volume is visually categorized on the displayed representation of the reconstructed 3D dataset.

20. The system of claim 17, wherein the processor is further configured to:
compare the blood flow speed to one or more predetermined ranges, one or more blood flow speed thresholds, or any combination thereof; and
determine the first color based on the comparison.
